(19) United States
(12) Patent Application Publication
(10) Pub. No.: US 2022/0079575 A1
(43) Pub. Date: Mar. 17, 2022

(54) EYE SPECULUM

(71) Applicant: GA HEALTH COMPANY LIMITED, Shek Mun (HK)

(72) Inventor: Kenneth McCabe, Shek Mun (HK)

(73) Assignee: GA HEALTH COMPANY LIMITED, Shek Mun (HK)

(21) Appl. No.: 17/021,486

(22) Filed: Sep. 15, 2020

(51) Int. Cl. A61B 17/02

(52) U.S. Cl. CPC A61B 17/0231; A61B 2017/00367; A61B 17/0206

(57) ABSTRACT

An eye speculum including a frame and a first and a second holder movably mounted on the frame. A first retraction blade is secured to the first holder and a second retraction blade secured to the second holder. An adjustment mechanism is operably secured to the frame and to the first and second holders. The adjustment mechanism includes a rotatable actuating device engageable by a user, wherein rotation of the actuating device translates the first and second holders relative to each other to control a separation distance between the first and second retractor blades.

### FIELD OF TECHNOLOGY

The present disclosure relates to an eye speculum and more particularly an eye speculum which can be easily adjusted.

### BACKGROUND

The eyelid speculum is a well known device in the field of ophthalmology, both for use in diagnostics and in surgery of the anterior and posterior segments of the eye. The purpose of an eyelid speculum is to mechanically retract the eyelids from the surface of the eyeball. For example, the eyelid is retracted when performing a phacoemulsification or LASIK procedure.

Unfortunately, lid speculums of the prior art do not operate in an anatomically correct manner. Prior art speculums tend to create pressure points that cause patient discomfort. For example, an eye speculum typically comprises a two-armed instrument with an adjusting device for adjusting the angle of one arm with respect to the other. Eye speculums typically have a retractor or blade at the distal end of each arm that may comprise a scoop-shaped piece in cross-section. Each scoop is inscribed around and under a respective eyelid, and the eye speculum holds the eyelids back when the adjusting device is adjusted to open the angle between the arms. By changing the angle between the arms to retract the eye list causes the eyelids to be opened at an angle which is an unnatural movement.

In addition, eye speculums typically require two hands to operate them. One hand holds the device in place which the other adjusts the distance between the blades.

Accordingly, it would be advantageous to provide a lid speculum that safely and easily exposes the eyeball to allow greater access thereto during eye surgery.

### SUMMARY

The present disclosure provides an eye speculum including a frame and a first and a second holder movably mounted on the frame. A first retraction blade is secured to the first holder and a second retraction blade secured to the second holder. An adjustment mechanism is operably secured to the frame and to the first and second holders. The adjustment mechanism includes a rotatable actuating device engageable by a user, wherein rotation of the actuating device translates the first and second holders relative to each other to control a separation distance between the first and second retractor blades.

The present disclosure also provides an eye speculum including a frame and handle depending from the frame. A first and a second holder is movably mounted on the frame. The first and second holders each have a linear gear portion. A first retraction blade is secured to the first holder and a second retraction blade secured to the second holder. An adjustment mechanism is operably secured to the frame. The adjustment mechanism is operably connected to the first and second holders for translating the first and second holders relative to each other. The adjustment mechanism includes a round gear rotatably secured to the frame and meshed with the linear gear portion of the first and second holder. Rotation of the round gear controls a separation distance between the first and second retractor blade and the separation distance is independent of an angular orientation between the first and second holder.

The present disclosure further provides a method of operating an eye speculum including obtaining an eye speculum. The eye speculum including a frame and a first and a second holder movably mounted on the frame. A first retraction blade is secured to the first holder and a second retraction blade secured to the second holder. An adjustment mechanism is operably secured to the frame and to the first and second holders. The adjustment mechanism includes a rotatable actuating device engageable by a user, wherein rotation of the actuating device translates the first and second holders relative to each other to control a separation distance between the first and second retractor blades. The method further including grasping the elongate handle in one hand and rotating the wheel with the thumb of the grasping hand to translate the first and second holders relative to each other.

### BRIEF DESCRIPTION OF DRAWINGS

FIG. 1 is a perspective view of the eye speculum of the present disclosure.

FIG. 2 is a front elevational view of the eye speculum.

FIG. 3 is a cross-sectional view taken along line 3-3 of FIG. 2.

FIG. 4 is a side elevational view of the eye speculum.

FIG. 5 is a detailed perspective view of a frame.

FIG. 6 is a perspective view of an adjustment mechanism actuating device.

FIG. 7 is a detail perspective view of the eye speculum with one holder removed.

FIG. 8 is a perspective view of a first side of a holder.

FIG. 9 is a perspective view of a second side of the holder.

FIG. 10 is a detail view of a retractor blade being placed on an arm.

FIG. 11 is a detail view of a retractor blade placed on an arm.

FIG. 12 is a detail exploded view of a holder removed from an arm.

FIG. 13 is a detail perspective view of a holder attached to an arm.

### DETAILED DESCRIPTION

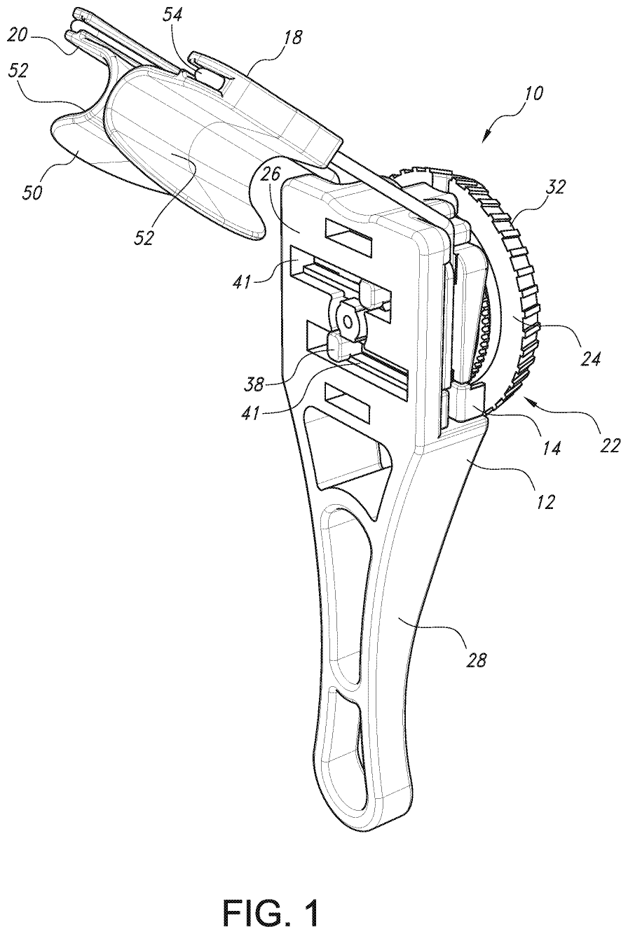

With reference to FIGS. 1-4, an eye speculum 10 is shown. The eye speculum 10 is used to retract a patient's eyelids in order to permit a practitioner to treat the eye. The speculum 10 includes a frame 12 which supports a first and a second holder 14, 16 movably mounted thereon. A first retraction blade 18 is secured to the first holder 14 and a second retraction blade 20 is secured to the second holder 16. The frame 12 also supports an adjustment mechanism 22 operably secured thereto. The adjustment mechanism 22 is operably connected to the first and second holders for translating the first and second holders 14, 16 relative to each other. The adjustment mechanism 22 includes an actuating device 24 engageable by a user to control a separation distance between the first and second retraction blade 18, 20.

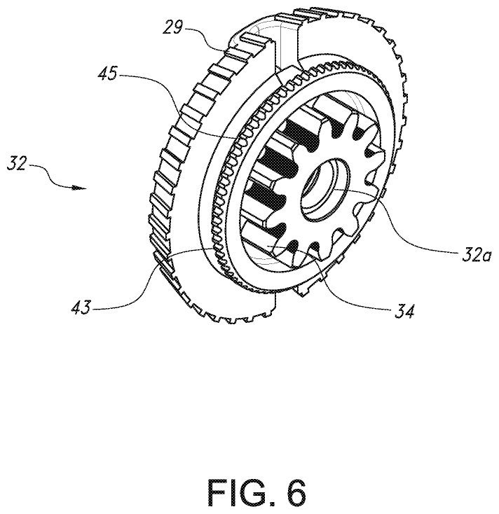

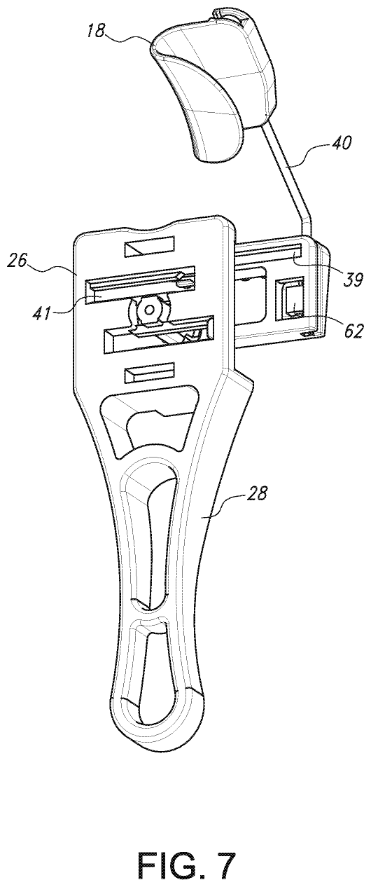

With additional reference to FIG. 6, the frame 12 may include a wall 26 extending upwardly from a base 27. An elongate handle 28 extends downwardly from the base 27. The handle 28 may have opposed concave sides 30 to provide a smooth surface to facilitate the holding of the eye speculum 10 by a practitioner. By way of example, the frame 26 and handle 28 may be formed of a plastic material.

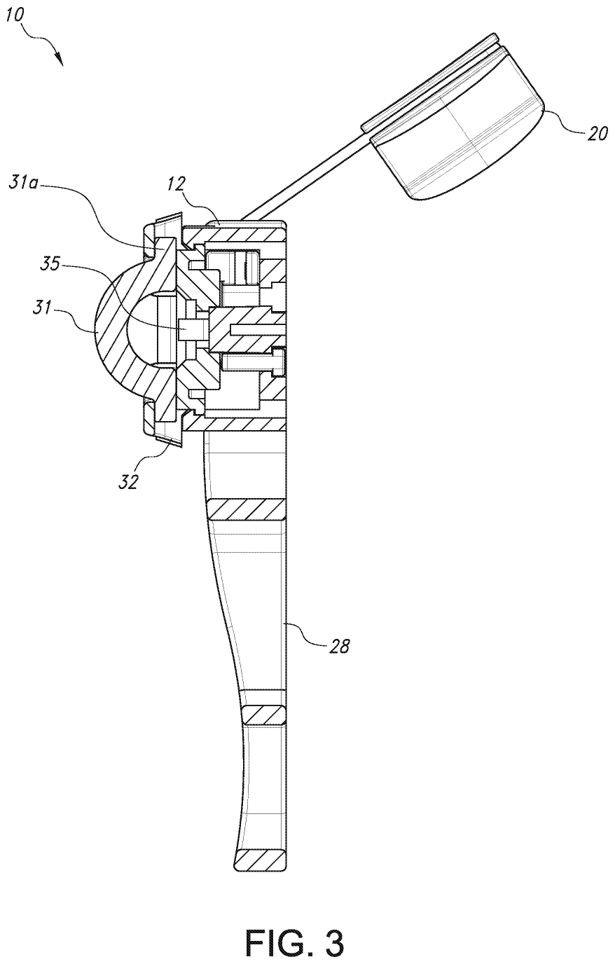

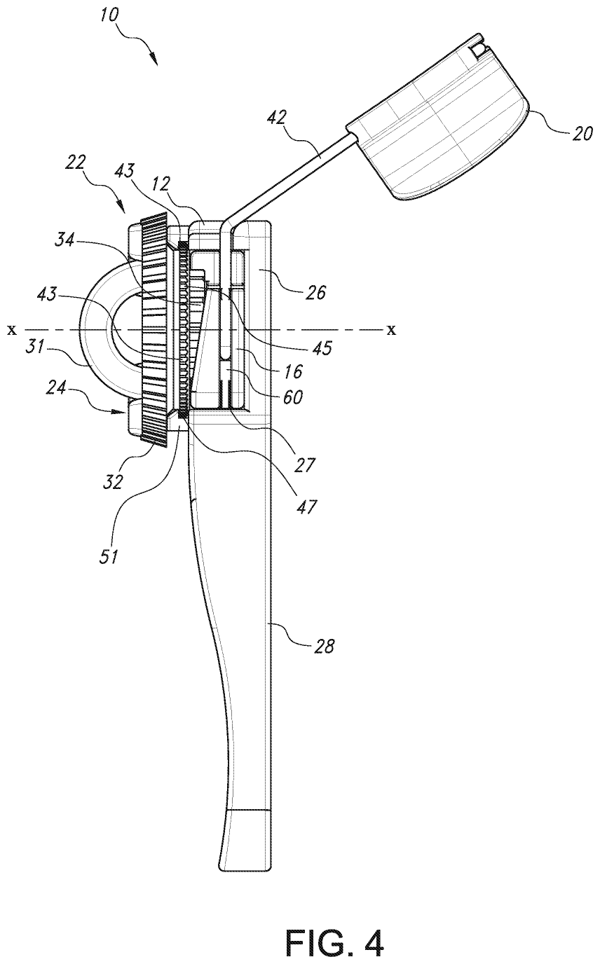

The retraction blades adjustment mechanism 22 supported on the frame 12 permits a practitioner to both hold the eye speculum 10 and adjust the distance between the retraction blades 18 and 20 using one hand. The adjustment mechanism actuating device 24 includes a wheel 32 rotatably supported on the frame wall 26. The wheel rotates about an axis X-X as shown on FIG. 4. The wheel 32 has a diameter larger that the width of the frame wall 26. The outer diameter may include a plurality of radially spaced ridges 29 to facilitate engagement by a user. Therefore, the wheel 32 extends beyond the frame 12, which makes the wheel easy to engage by a user. The wheel 32 may include a U-shaped grip 31 with outwardly extending ends 31a pivotally secured to the wheel. The grip 31 may pivot between a storage position wherein it lies flat against the wheel 32 and an extended position where it extends perpendicularly from the wheel. In the extended position, shown in FIGS. 3 and 4, the grip 31 provides an alternative way for a user to engage and turn the wheel 32.

With reference to FIGS. 3-6, the wheel 32 includes a round gear 34 having circumferentially spaced teeth. A post 33 extending from the frame wall rotatably connects the wheel 32 and round gear 34 to the frame 12. The end of the post may include reliant fingers 35 that project radially outwardly. The fingers 35 deflect inwardly to permit the wheel central opening 32a to pass onto the post 33 and then return to the original position to retain the wheel 32 on the post 33.

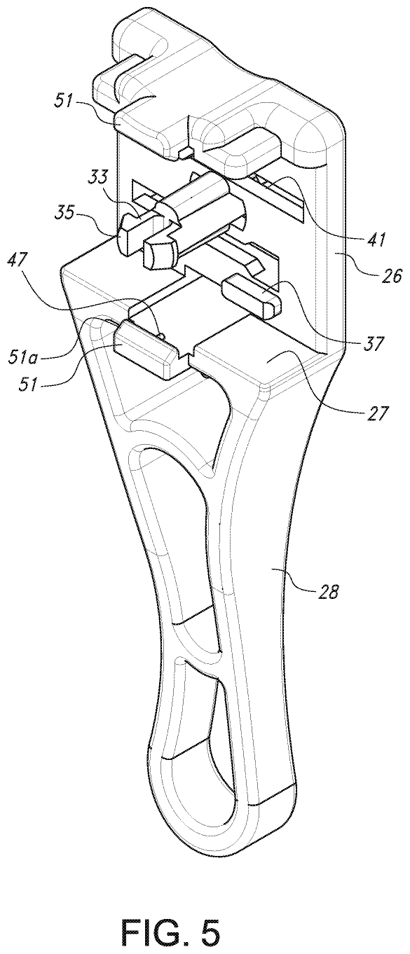

With reference to FIGS. 4 and 6, the wheel 32 may further include a detent ring 43 formed therein which includes a number of indentations 45 formed therein. The detent ring projects outwardly forming a lip 49. Resilient arms 51 with catches 51a project from the frame wall 26 and engage the detent ring 43 which helps to retain the wheel 32 on the frame 12. The indentations 45 engage a raised protrusion 47 (FIG. 5). The engagement between the protrusion 47 and the indentations 45 results in the position of the wheel 32 being retained in a selected position. The engagement can overcome by a user rotating the wheel 32 to adjust the position of the retraction blades 18, 20.

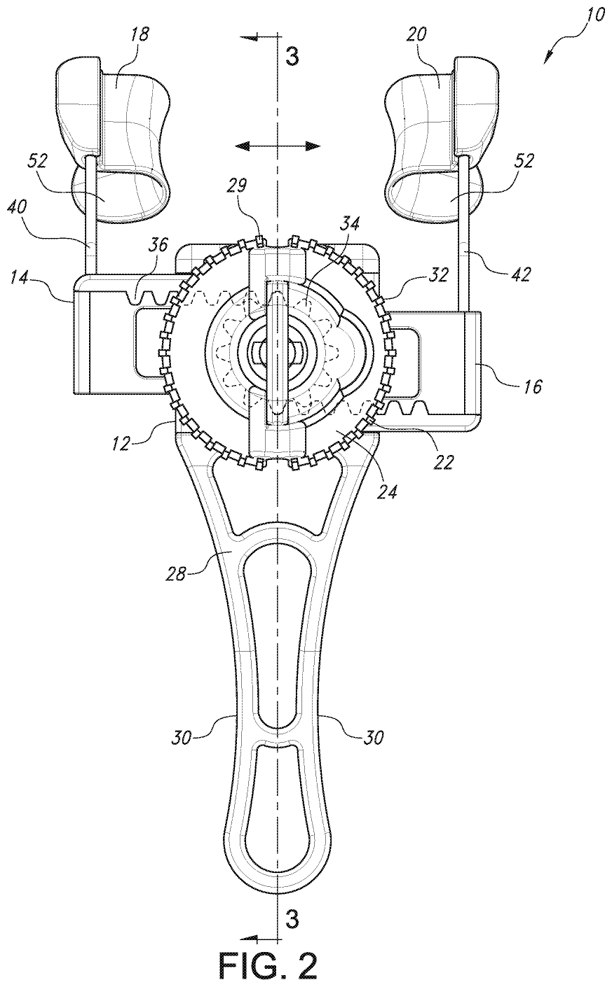

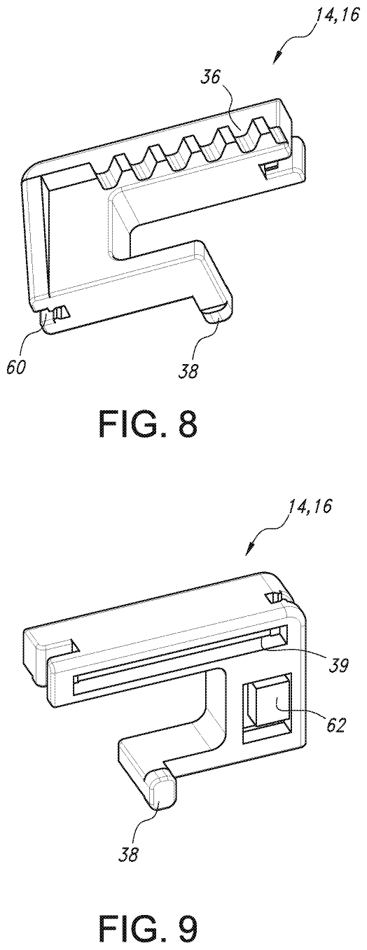

With reference to FIGS. 2, 8 and 9, the first and second holders 14, 16 are translatably supported on the frame 12 such that they can move in a linear direction relative to each other, as shown by the arrow in FIG. 2. The first and second holders 14, 16 have a generally U-shaped configuration with one of the arms of the U have a portion including a linear gear, or rack 36. The linear gears 36 mesh with the teeth of round gear 34 such that rotation of the wheel 32 causes the holders 14, 16 to move in opposite linear directions. For example, when the wheel 32 is rotated clockwise, the first holder 14 will translate in a first direction and the second holder 16 will translate in a second direction, such that the retraction blades 18, 20 move away from other. The first and second directions are opposite each other. When the wheel 32 is rotated counterclockwise, the first and second holders 14, 16 will move in the opposite directions such that the retraction blades 18, 20 move toward each other.

The first and second holders 14, 16 may be interchangeable with each other and similarly formed. This simplifies manufacturing since the same piece can be used for both the first and second holder. When secured to the frame 12 the first holder 14 is in a first orientation and the second holder 16 is in a second orientation. When in the first and second orientation, the first and second holders 14, 16 are inverted with respect to each other as shown in FIG. 2.

With reference to FIGS. 5 and 9, the frame wall and holders includes structures in order to guide the motion of the first and second holders and the retraction blades thereon. Specifically, frame wall 26 includes a pair of spaced linear projections 37 formed therein. The first and second holders 14, 16 each have an elongate slot 39 formed therein which receives one of the projections 37 extending from the frame wall 26. In addition, each of the first and second holders 14, 16 may include a lug 38 extending therefrom. The lug 38 extends into a linear channel 41 formed in the frame wall 26. The cooperation between the slots 39 and projections 37 and the lugs 38 and the channels 41 guide the first and second holders 14, 16 as they translate relative to each other.

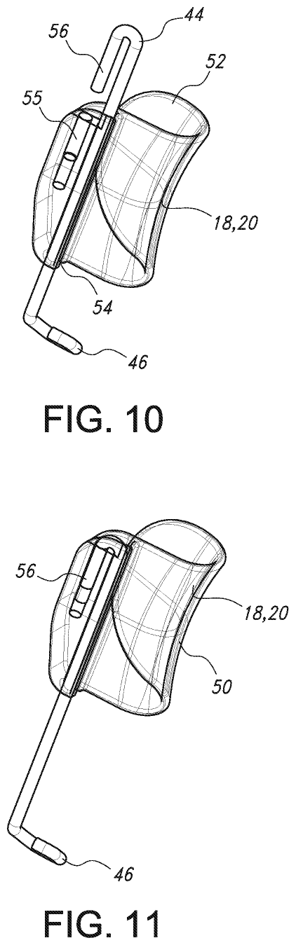

With reference to FIGS. 10-13, extending from the first holder 14 is a first arm 40 and extending from the second holder 16 is a second arm 42. The arms 40, 42 extend upwardly and outwardly from the frame 12. The first and second arms 40, 42 terminate in the retraction blades 18 and 20, respectively. Each of the first and second arms has a proximal end secured to the holder and a distal end secured to the blade. The arms 40, 42 may be rigid, wire-like members having hooked distal ends 44 to facilitate attaching the blades 18, 20 and hooked proximal ends 46 to facilitate securement to their respective holders 14, 16 as will be described below.

With reference to FIGS. 10 and 11, the first and second retraction blades 18, 20 are similarly formed and each include curved bodies 50 defining an open channel 52. Then the first and second blades are secured to the arms, the open channels are opposed from each other and face opposite directions. The blades may be formed of a soft resilient material such as nylon or polypropylene. The first and second retraction blades are shaped such that they can engage the upper and lower eyelids and permit the eyelids to be separated from each other when the practitioner operates the wheel 32. With the eyeball exposed, the practitioner can perform the requisite treatment.

As shown in FIGS. 10 and 11, the first and second retraction blades 18, 20 each have a groove 54 for receiving the distal end of the arm. The blades further include a bore 55 in which the tip 56 of the hook is inserted. This permits the blades to be easily attached to the arms and prevents them from rotating relative to the arm.

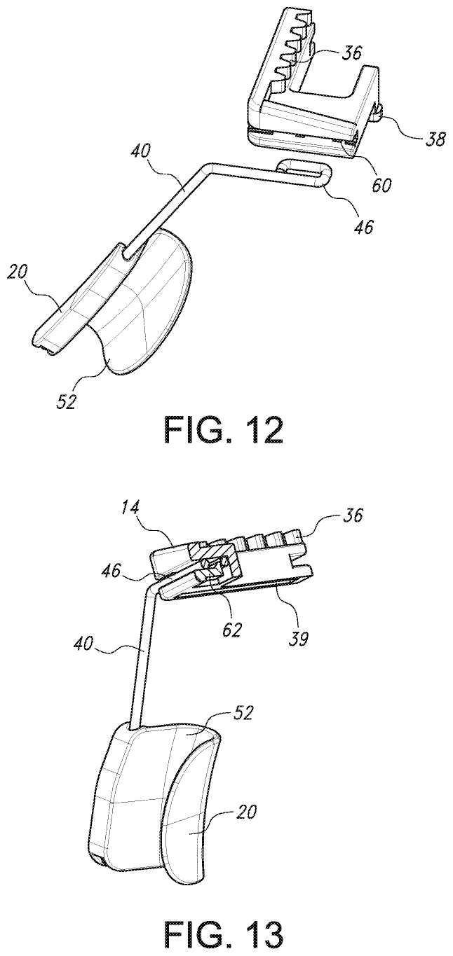

With reference to FIGS. 12 and 13, the proximal end 46 of the first and second arms each are secured to their respective holders 14, 16 by a recess 60 formed in the holders. A resilient clip 62 extends in a cantilevered manner from the holder into the recess 60. The clip 62 may be integrally formed with the holder to which it is attached. The recess 60 receives the proximal end of the arm 66 and the insertion of the proximal deflects the clip 62. When the arm passes then clip 62 it returns to an undeflected position thereby retaining the arm in the holder.

Due to the linear translation of the holders and retractor blades attached thereto, the separation distance between the first and second retractor blades is independent of the angular orientation of the first and second holders. Therefore, the angular orientation between the first and second retractor blades does not change as the distance between the blades is adjusted. The linear separation of the blades mimics the natural linear movement of the eye lids.

In operation, a practitioner using the eye speculum 10 may first rotate the wheel 32 to bring the first and second retraction blades 18 and 20 together such that they are spaced a distance that permits them to be inserted under the eyelids. The practitioner, while holding the eye speculum by the handle 28, then inserts the first and second retraction blades 18 and 20 into position around the upper and lower eyelids. The practitioner then rotates the wheel 32 with their thumb, of the same hand holding the handle 28, in a direction which causes the distance between the blades 18, 20 to be increased and the eyelids retracted. The practitioner may then treat the eyes as necessary.

When the procedure is completed, the practitioner may then turn the wheel 32 in the opposite direction to decrease the distance between the blades 18, 20, thus permitting the eyelids to return to their initial state. The eye speculum 10 can then be removed from the patient.

Given the teachings provided herein, one of ordinary skill in the art will be able to contemplate other implementations and applications of the techniques and disclosed embodiments. Although illustrative embodiments have been described herein with reference to the accompanying drawings, it is to be understood that illustrative embodiments are not limited to those precise embodiments, and that various other changes and modifications are made therein by one skilled in the art without departing from the scope of the appended claims.

What is claimed is:

1. An eye speculum comprising:
a frame;
a first and a second holder movably mounted on the frame;
a first retraction blade secured to the first holder and a second retraction blade secured to the second holder;
an adjustment mechanism operably secured to the frame and to the first and second holders; and
the adjustment mechanism including a rotatable actuating device engageable by a user, wherein rotation of the actuating device linearly translates the first and second holders relative to each other to control a separation distance between the first and second retractor blades.

2. The eye speculum as defined in claim 1, wherein the actuating device is connected to a gear and the first and second holders each include a linear gear operatively engaged with the gear, wherein rotation of the actuating device translates the first holder and second holder relative to each other and changes the separation distance between the first and second blade.

3. The eye speculum as defined in claim 2, wherein the separation distance between the first and second retractor blade is independent of the angular orientation of the first and second holder.

4. The eye speculum as defined in claim 1, wherein the first and second retraction blades each include curved bodies defining a channel opening and the channel openings of the first and second retraction blades facing in opposite directions.

5. The eye speculum as defined in claim 1, wherein the frame includes a pair of guide elements and each one of the pair of guide elements being engageable with one of the first and second holder for guiding the linear translation of the first and second holders relative to the frame.

6. The eye speculum as defined in claim 5, wherein the pair of guide elements each include a slot formed in the frame and the first and second holders include projections each disposed in one of the slots.

7. The eye speculum as defined in claim 1, wherein the first retraction blade is secured to the first holder by a first arm and the second retraction blade is secured to the second holder by a second arm.

8. The eye speculum as defined in claim 7, wherein the first blade has a groove for receiving a distal end of the first arm, and the second blade has a groove for receiving a distal end of the second arm.

9. The eye speculum as defined in claim 8, wherein the distal end of the first and second arms each have a hook that facilitates connection with one of the first and second blade retractors.

10. The eye speculum as defined in claim 9, wherein the first and second blade each have a bore disposed adjacent the groove, a portion of the arm lying in the groove and a tip portion being disposed in the bore to retain the holder to the arm.

11. The eye speculum as defined in claim 9, wherein the first and second arms each have a proximal end secured to their respective holder.

12. The eye speculum as defined in claim 11, wherein the first and second holders each have a recess formed therein and a resilient clip disposed in the recess, the recess receiving the proximal end of the arm and the insertion of the proximal deflects the clip and the clip retains the proximal end therein.

13. The eye speculum as defined in claim 1, wherein the first holder and second blade holder are similarly formed and interchangeable with each other.

14. An eye speculum comprising:
a frame;
a handle depending from the frame;
a first and a second holder movably mounted on the frame, the first and second holders each having a linear gear portion;
a first retraction blade secured to the first holder and a second retraction blade secured to the second holder;
an adjustment mechanism operably secured to the frame, the adjustment mechanism operably connected to the first and second holders for translating the first and second holders relative to each other; and
the adjustment mechanism including a round gear rotatably secured to the frame and meshed with linear gear portion of the first and second holder, wherein rotation of the round gear controls a separation distance between the first and second retractor blade and the separation distance is independent of an angular orientation between the first and second holder.

15. The eye speculum as defined in claim 14, wherein the round gear is operably connected to a rotatable actuating device.

16. The eye speculum as defined in claim 15, wherein the round rear is on an opposite side of the frame as the rotatable actuating device

17. The eye speculum as defined in claim 14, wherein the rotatable actuating device is a wheel having a diameter larger that a width of the frame.

18. The eye speculum as defined in claim 14, wherein the frame includes a pair of guide elements and each one of the pair of guide elements being engageable with one of the first and second holder for guiding the linear translation of the first and second holders relative to the frame.

19. A method of operating an eye speculum comprising:
obtaining an eye speculum as defined in claim 1;
grasping the elongate handle in one hand; and
rotating the wheel with the thumb of the grasping hand to translate the first and second holders relative to each other.

20. The method as defined in claim 18, placing the first and second retraction blades around a patient's eye lids.
